(19) United States
(12) Patent Application Publication
(10) Pub. No.: US 2012/0197086 A1
(43) Pub. Date: Aug. 2, 2012

(54) MEDICAL VISUALIZATION TECHNIQUE AND APPARATUS

(75) Inventors: Sean Morris, Co. Roscommon (IE); Roger Mecca, Corona Del Mar, CA (US)

(73) Assignee: Nellcor Puritan Bennett LLC, Boulder, CO (US)

(21) Appl. No.: 13/018,006

(22) Filed: Jan. 31, 2011

(51) Int. Cl. A61B 1/267

(52) U.S. Cl. 600/188

(57) ABSTRACT

According to various embodiments, an airway visualization device may facilitate the selection and insertion of airway devices. The device may allow clinicians to more effectively determine the appropriate diameter tracheal tube for a particular patient, which in turn may decrease work of breathing and/or discomfort for the patient. In particular embodiments, the airway visualization device provides a displayed image of the patient's airway upon which a representative tracheal tube or other airway device may be superimposed. The clinician may evaluate the superimposed image to determine if the representative tracheal tube is appropriately sized for the airway.

### BACKGROUND

The present disclosure relates generally to medical devices and, more particularly, to techniques for visualizing a patient vessel or airway to facilitate the insertion and placement of in-dwelling medical devices.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

For example, tracheal tubes may be used to control the flow of air or other gases through a patient's airway. Such tracheal tubes may include endotracheal (ET) tubes, tracheostomy tubes, transtracheal, and endobronchial tubes, etc. In many instances, it is desirable to provide a seal between the outside of the tube or device and the interior of the passage in which the tube or device is inserted. In this way, substances can only flow through the passage via the tube or other medical device, allowing a medical practitioner to maintain control over the type and amount of substances flowing into and out of the patient.

The proper insertion and placement of devices within a patient's airway may be complex. Often, a clinician may observe a patient's physical characteristics, such as age, gender, size, and/or weight, and use these characteristics to estimate the size of the patient's airway and, in turn, the appropriately-sized tracheal tube. However, certain patients may have trachea or other airway abnormalities that may not be predicted by observation of external characteristics and that may influence the sizing of an inserted device. In certain situations, insertion of a tracheal device may be aided with visualization of the trachea performed during laryngoscopy. During an intubation procedure, a practitioner may employ a lighted laryngoscope during introduction of a tracheal tube. However, a clinician may have difficulty translating images from direct visualization or from a laryngoscope to a real-world setting.

### BRIEF DESCRIPTION OF THE DRAWINGS

Advantages of the disclosed techniques may become apparent upon reading the following detailed description and upon reference to the drawings in which:

FIG. 1 is a block diagram of an airway visualization system to be used in conjunction with an airway device, in accordance with an embodiment of the present technique;

FIG. 2 is a flow diagram of an airway visualization method in accordance with an embodiment of the present technique;

FIG. 3 illustrates a display of a patient airway with a representation of a tracheal tube of a predetermined size;

FIG. 4 is a flow diagram of airway visualization method for selecting a tracheal tube for a challenging airway in accordance with an embodiment of the present technique;

FIG. 5 illustrates a display of a patient airway with a representation of a tracheal tube that is too large for the depicted airway;

FIG. 6 illustrates a display of a patient airway with a representation of a tracheal tube that is too small for the depicted airway; and

FIG. 7 is a block diagram of a closed-loop system of cuff inflation based on the airway visualization device.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Insertion of an airway device typically involves the intervention of skilled medical personnel. An experienced clinician may simply observe a patient, taking into account factors such as a patient's size and clinical condition, and form an estimate of the size of the patient's airway. In selecting an appropriately-sized device for the patient, clinicians attempt to match the size of any inserted airway device with the size of the airway. For example, it is desirable to maximize the diameter of a tracheal tube used for mechanical ventilation to decrease the work of breathing for the patient. When the tracheal tube is too small, the patient has to work harder to push air out of the lungs and through the tube during exhalation. An appropriately-sized tracheal tube may result in decreased work of breathing relative to a tube that is too small for the patient's airway.

However, because insertion of an airway device is complex, clinicians may hesitate to select a tracheal tube that may be too large to avoid having to restart the intubation procedure if the tube cannot be advanced into the airway. In such cases, clinicians may be biased towards selecting tubes that are too small, as these tubes are more likely to result in successful intubation. Further, because the size of the patient's airway is typically estimated and is not based on a direct assessment of the airway itself, these estimates may be inaccurate for the airways of atypical patients, resulting in a mismatch between the size of the airway device and the size of the airway. In addition, for airway devices that are uncuffed, i.e., that do not include an inflatable cuff that seals the airway, the tube external diameter is related to its sealing performance. A tube that is too small may result in ventilation leaks past the tube, which may have a negative impact on ventilation.

As described in detail below, embodiments of a system for visualizing a patient airway are provided herein. In particular, embodiments of the present disclosure relate to a system for determining an airway size based on image or other information collected from the airway, such as information provided by a laryngoscope or a camera associated with an airway device. The assessment of the patient's airway may be used to select au appropriately-sized airway device. In one embodiment, scaled images of tracheal tubes of various sizes may be superimposed on an airway image to allow a clinician to select the best match. In certain embodiments, it is envisioned that the matching of the airway image to the scaled images or representations of the tracheal tubes may include manual matching or assessment steps, i.e., steps performed by a clinician. In particular, in contrast to more complex signal processing arrangement in which an image may undergo segmentation or other types of processing in order to pick out the features that define the borders of an airway, embodiments of the present techniques do not require complex and/or processor-intensive algorithms. The disclosed visualization system may provide the advantage of providing a display interface that is easy for a clinician to maneuver and understand. In addition, by allowing the clinician to manipulate the images and select an appropriately-sized tube, the present techniques allow for variability in clinical judgment while providing an additional degree of confidence for the clinician. In other embodiments, the image information may be automatically processed to determine the tracheal diameter or size. In yet another embodiment of the present technique, the tracheal diameter information may be used to control inflation of a cuff. That is, given a particular tracheal diameter, a desired inflation volume for a cuff may be selected.

The airway visualization system as provided may be used for selecting appropriately-sized airway devices for insertion into a patient. For example, the airway devices may include tracheal tubes, endotracheal tubes, tracheostomy tubes, endobronchial tubes, or any medical devices for insertion into a patient's airway. Further, in particular embodiments, the techniques provided herein may be used to assess the size and/or condition of any patient vessel or passage, including vascular passages, GI passages, and reproductive passages and may be used in conjunction with any suitable indwelling or inserted medical device, including catheters, stents, sensors, or drug-delivery devices.

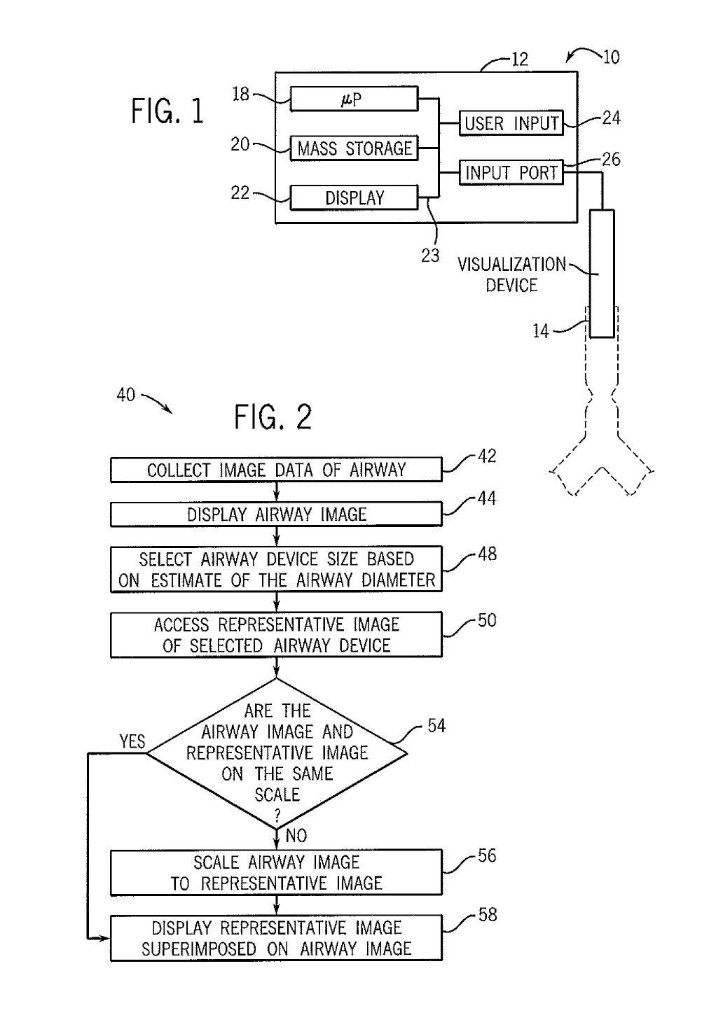

Turning now to the figures, FIG. 1 depicts an embodiment of an airway visualization system 10 that includes a monitor 12 configured to be used in conjunction with a visualization device 14. In one embodiment, the visualization device 14 may be any suitable device for gathering image data of a patient's airway, including the trachea. While the visualization device 14 may be external to the patient, it is envisioned that the visualization device 14 may also inserted directly into the patient airway, as shown in FIG. 1, either prior to or concurrently with an airway device, to gather data that may be sent to the monitor 12 for further processing. For example, the visualization device 14 may be a bronchoscope, laryngoscope or a camera coupled to another inserted medical device. Because the techniques provided herein relate to determining a size and shape of a patient's airway, an inserted visualization device 14 may be able to gather image data that may be directly scaled without complex processing.

The monitor 12 includes a processor 18 for executing routines or instructions stored in mass storage 20, such as instructions for implementing the techniques discussed herein and instructions associated with the display and manipulation of visualization data collected by the visualization device 14. Additionally, the monitor 12 may include a display 22 coupled to the processor 18 via internal bus 23 and configured to display information regarding the output generated by the visualization device 14, such as images of a patient's airway, e.g., a trachea. The display 22 may also be used for display of other information, e.g., information related to a tracheal size or diameter, according to the inputs provided by the user, and the monitor 12 may include various input components 24, such as knobs, switches, keys and keypads, touchscreens, buttons, etc., to provide for operation and configuration of the monitor 12. The monitor 12 may also include an input port 26 for coupling to the visualization device 14. For example, the computer may include a USB port for coupling to an external device. In other embodiments, the monitor 12 may include a transceiver for coupling to wireless medical devices.

The monitor 12 may be capable of providing indications related to the selected tube size. For example, as provided herein, a user, e.g., a clinician, may select an image that is associated with a tube of a particular size, e.g., a commercially available tracheal tube. After the clinician fits the image of the tube onto the image of the trachea, information associated with the selected tracheal tube, such as its outer diameter, inner diameter, and other identifying information, may be displayed. In addition, in embodiments in which all or at least a portion of the fitting is performed automatically, the monitor 12 may provide a graphical, visual, or audio representation of a proper fit or an improper fit. For example, an indicator associated with an appropriately-sized tracheal tube for the trachea displayed may include green light indicated on a display or a short tone generated by a speaker associated with monitor 12. Similarly, an indicator associated with a poor fit may trigger an alarm, which may include one or more of an audio or visual alarm indication. In one embodiment, the alarm may be triggered if the outer diameter of the selected tracheal tube is larger than an estimate of the tracheal diameter, or if the selected tube is sufficiently large to impinge upon an abnormality within a patient's trachea (e.g., such as a tracheal stenosis, tracheomalacia, A-V malformation, etc.).

FIG. 2 is a process flow diagram illustrating a method 40 for visualizing a patient's airway. The method may be performed as an automated procedure by a system, such as a system 10 that includes the monitor 12 and the visualization device 14. For example, certain steps may be performed by a processor, e.g., processor 18, that executes stored instructions for implementing steps of the method 40. In addition, in particular embodiments, certain steps of the method 40 may be implemented by a clinician.

According to a particular embodiment, the visualization device 14 collects image data of the patient's airway at step 42 and provides data to the monitor 12. The monitor 12 accesses the image data and displays an image of the airway on the display 22 at step 44. A clinician may then manipulate the airway image in various ways. For example, the clinician may view the image and develop an estimate of the airway diameter, or take note of any particular restrictions or physiologic abnormalities. The clinician may then provide an input to the monitor 12 related to the airway diameter estimate at step 48. In one embodiment, the input relates to the selection of an airway device that is appropriately-sized for the airway based on an estimate of the airway diameter. The monitor 12 accesses a representative image of the selected airway device at step 50. At step 54, the relative scales of the airway image and the representative airway device are assessed, and a scaling step may be performed at step 56 if the airway image and the representative tracheal tube 64 are not already on the same scale before proceeding. When the airway image and the representative tracheal tube are on the same scale, the method proceed to step 58 and the representative image is superimposed onto airway image.

By viewing the representative image superimposed on the airway image, the clinician may make determinations as to whether the tube represented is appropriately sized for the airway. In one embodiment, the clinician may determine that the selected representative image represents a tube that is appropriately sized for the patient. Based on the determination, the clinician may provide an input related to the selection, such as storing the appropriate tube size and/or the image of the representative image superimposed on the airway image in the mass storage 20 of the monitor 12. In another embodiment, if the representative image is too small, the clinician may select a representative image associated with a larger tube size to better match the airway. Selection of another representative image may trigger removal of the previous representative image from the display, and replacement of the new image. In yet another embodiment, if the representative image is too large, the clinician may select a representative image associated with a smaller tube size.

Further, the clinician may provide inputs to the monitor 12 related to information on the sizing of particular tubes, the success of intubation with the selected tube size, or observed airway anomalies or anatomical features. For example, the information may be related to determinations that a particular tube size appears to be too large or too small. Other users may access this stored information when inserting airway devices at later points. In addition, the displayed images may be a side-by-side comparison of an airway image with superimposed representative images of different sizes. In another embodiment, a clinician may view display of the airway image with different sizes of representative images displayed over time, i.e., a user input may cause the monitor 12 to scroll through or otherwise change the size of the representative image that is displayed on the airway image for comparison. It should be understood that, in specific embodiments, the steps of selecting the representative image, determining an airway size, and/or determining an appropriate tracheal tube size based on the airway image data may be automatic, i.e., performed by the monitor 12. For example, based on one or more of default settings, patient information, or image analysis, the monitor 12 may select a representative image for superimposition on the airway image. In one embodiment, for patients of a certain size or weight, the monitor 12 selects a default representative image that corresponds to tracheal tubes known to fit such patients. After automatic selection of the representative image, the monitor 12 may superimpose the selected representative image on the airway image for further assessment. The assessment may be performed by the clinician or the monitor 12 to determine if the fit is acceptable or poor. If the assessment is automatic, the monitor 12 may provide indications related to the fit and/or may automatically select another representative image for assessment until an acceptable fit is achieved.

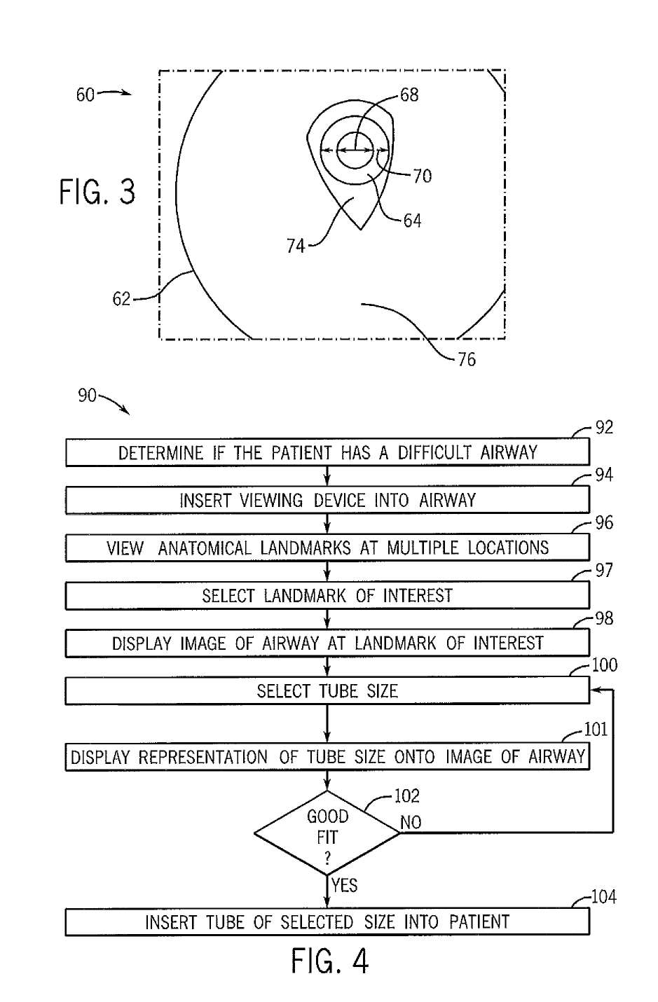

FIG. 3 is an example of a display image 60 that includes an airway image 62 on which a representative tracheal tube 64 has been superimposed. The airway image 62 may represent a top down image showing a pathway of the trachea surrounded by tissue. The representative tracheal tube 64 is an annulus having an inner diameter 68 and an outer diameter 70. As noted, the representative tracheal tube 64 may be selected by the clinician after viewing the airway image 62. In other embodiments, the selection of the representative tracheal tube 64 may be automatic and may be accomplished via various image processing modalities, such as segmentation. For example, the airway image 62 or the data associated with the airway image 62 may be processed to determine the shape and/or size of the airway region 74 surrounded by relatively lighter tissue 76. As shown, the airway region 74 has an irregular shape. In such cases, determining the size of the airway region 74 may also include a determination of the largest annulus that may fit within the airway region 74. After determining a diameter of the trachea shown in the airway image 62, the monitor 12 may then select the best fit of a representative tracheal tube 64 and automatically superimpose the selected size representative tracheal tube 64 on the airway image 62. Depending on an input or decision step, the computer 12 may access the appropriate representative tracheal tube 64 for display on the airway image 62. The clinician may, however, in certain embodiments override any automatic selection of the tracheal tube 64 (or other indwelling medical device) based on clinical judgment.

It is envisioned that the user may be able to select from a library of representative tracheal tube 64 images. This library may be accessible to the user via a menu or other selection mechanism. In one embodiment, the displayed user interface may include icons representing respective representative tracheal tubes 64 of different sizes (e.g., different outer and inner diameters). In a particular embodiment, the user may drag and drop the representative tracheal tube 64 into place on the airway image 62. In addition, while the monitor 12 may execute instructions for superimposing the representative tracheal tube 64 onto the airway image 62, the relative position of these images may also be subject to user input. For example, the user may manipulate (e.g., via input components 24) the position of the representative tracheal tube 64 relative to the airway image 62 to achieve the appropriate positioning in the airway.

In particular embodiments, the representative tracheal tube 64 includes a representation of a cross-sectional image of a main lumen of a tracheal tube. The cross-section may be in the form of an annulus having an inner diameter and outer diameter associated with a standard tracheal tube size. For example, the representative tracheal tube 64 may represent tubes with inner diameters between 2-10.5 mm and outer diameters between 3-15 mm. Typically such tracheal tubes are sized according to the inner diameter of the tube and are provided in increments of 0.5 mm (e.g., 4.0 mm, 4.5 mm, etc.), although other increments may be used. It is contemplated that the monitor 12 may be capable of displaying a plurality of representative tracheal tube images 64, each associated with a different tracheal tube size having a particular outer diameter and a particular inner diameter. For example, a particular representative tracheal tube 64 may have an outer diameter of 10.8 mm and an inner diameter of 6.5 mm. A different representative tracheal tube 64 may have an outer diameter of 9.4 mm and an inner diameter of 5.0 mm. It should be understood that, in one embodiment, the system 10 may include a library of images of representative tracheal tubes 64 that are representative of the standard sizes of tracheal tubes available from one or more vendors, e.g., from Nellcor Puritan Bennett LLC. In addition, the representative tracheal tube 64 may include a cross-sectional image representative of a dual-lumen tube, such as a Broncho-Cath™ tube. In such embodiments, the representative image may have a particular outer diameter, or particular outer and inner profile that encompasses two lumens. Further, it is envisioned that the representative tracheal tube 64 may be an irregular shape representing tubes having non-annular cross-sectional profiles, and images of such profiles may be provided as a library and capable of manual or automatic selection as described herein.

In particular, because the superimposed image may inform a clinician how a tracheal tube of a given size or shape may fit within the airway, the scales of both images may be approximately equal. That is, if the airway image 62 is scaled too large relative to the superimposed representative tracheal tube 64, the clinician may not gain an accurate understanding of the match between the represented tracheal tube and the airway. In one embodiment, the airway image 62 and the representative tracheal tube 64 are scaled to actual size. The scaling may be accomplished by any suitable image processing modality, including the use of anatomical or camera-specific landmarks (e.g. a scaling or dimension indicator present on the lens or viewing field) or embedded scaling features on the airway image. For example, in one embodiment, both the represented tracheal tube 64 and the airway image 62 may have scaling icons or indicators that may be aligned with one another to assess the correct scaling on the images. In addition, the scaling may also be accomplished via user input or manipulation. For example, one or both of the airway image 62 and the representative tracheal tube 64 may be scaled by the user, who may change the size of one or both of the images (e.g., via zooming in or out or by manipulating the images via a user interface) until the scales are approximately equal.

While the airway visualization techniques as provided may be used for insertion of an airway device into any patient in need thereof, it is envisioned that, in certain embodiments, the airway visualization system 10 may be used in for insertion of these devices into patients with typical or atypical airways. For example, patients with carcinoids or other protrusions within the trachea may be difficult to intubate. FIG. 4 is a flow diagram for a method 90 of intubating a patient with an atypical airway. At step 92, the patient is identified as having a difficult airway, for example via previously acquired medical information, e.g., a previous diagnosis or via imaging data, or from information gathered from the visualization device 14. At step 94, the visualization device 14 is inserted into the airway, and at step 96 image data is acquired at various locations within the airway. The clinician may select the smallest or most difficult portion of the airway at step 97 and capture a display still image of that portion for display at step 98.

As noted, certain embodiments of the present techniques incorporate steps that may be accomplished by the clinician. In one embodiment, the clinician may select from a menu a desired tube size to superimpose onto the airway image 62. In addition, the clinician may manipulate the displayed representative tracheal tube 64 relative to the displayed airway image 62 to position the representative tracheal tube 64 within the airway. By allowing the clinician to perform these steps manually, the monitor 12 may not require automatic steps for identifying the airway portion of the airway image 62 and fitting the tube within the identified airway. This may provide the advantage of requiring less processing power to be built into the monitor 12. However, it should also be understood that the estimate of the airway size and/or position and the determination of tracheal tube fit may, in certain embodiments, be at least partially performed by the monitor 12.

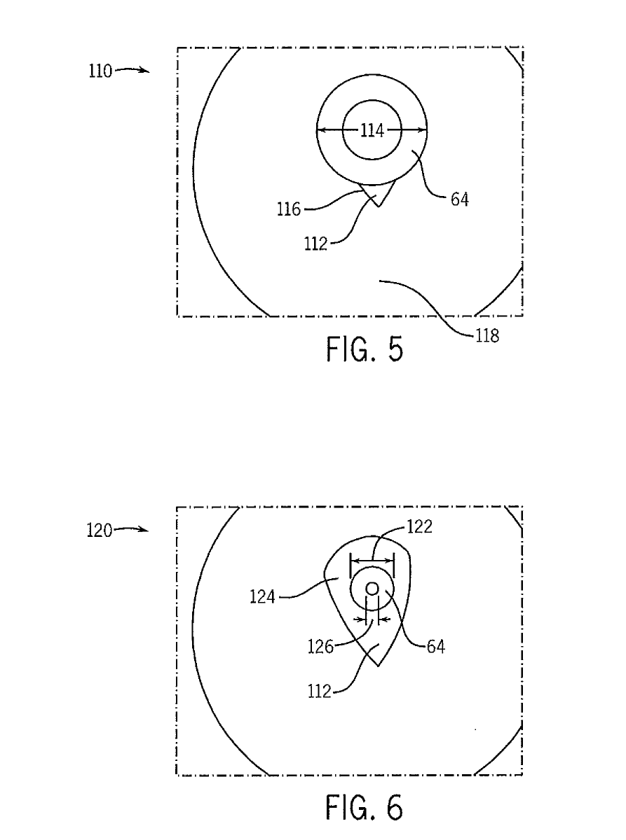

Based on the clinician's estimate of the size of the airway in the displayed image, the clinician may provide an input to the monitor 12 to select a representative tracheal tube 64 that will fit within the airway at step 100. The monitor 12 then displays the selected representative tracheal tube 64 superimposed on the airway image at step 101. At step 102, the clinician assesses the displayed image to determine if the fit is appropriate. For example, as shown in FIG. 5, the displayed image 110 may show that the representative tracheal tube 64 is too large for the airway 112. In the depicted example, the outer diameter 114 of the representative tracheal tube 64 diameter is predicted to extend beyond the edges 116 of the tracheal and into the tissue 118. This may be assessed by a visual inspection of the display 110. Similarly, FIG. 6 is an example of a display 120 in which the representative tracheal tube 64 diameter is smaller than the target the airway 112. The representative tracheal tube 64 at its outer diameter 122 leaves a gap 124 between the edges of the trachea and the representative tracheal tube 64. Although the representative tracheal tube 64 may fit within the airway, the inner diameter 126 may be relatively small and result in an increase in work of breathing for the patient, and thus not be optimal. Based on an assessment of the display 120, the clinician may determine that the representative tracheal tube 64 is too small. In such embodiments, in which the selected representative tracheal tube 64 is too big or too small, the method 90 may return to step 100 to select a different representative tracheal tube 64 so that the fit can be assessed again. Once an assessment of a good fit is made, the clinician may then use the information to select an appropriately-sized tracheal tube for insertion into the patient at step 104.

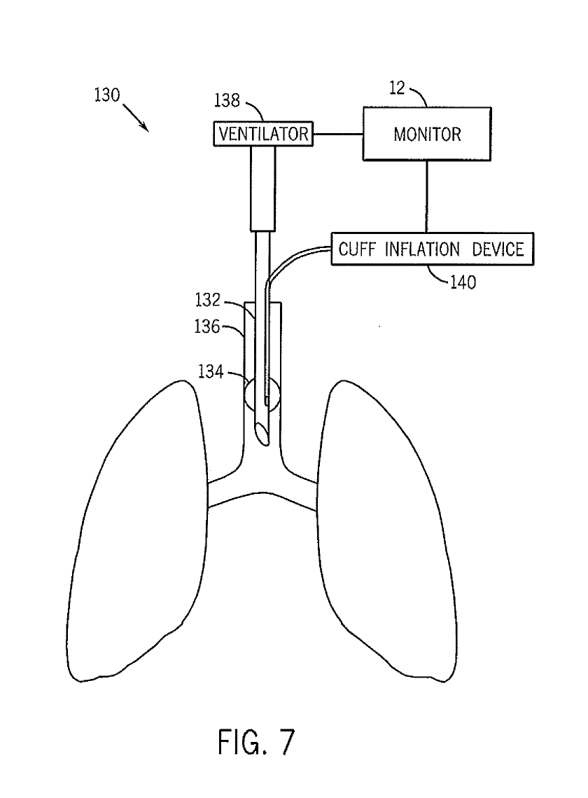

In addition to determining whether a tracheal tube or other airway device is appropriately sized for a patient, the airway visualization system as provided may allow a more accurate determination of the inflation volume for an inflatable cuff associated with the tracheal tube. FIG. 7 shows an exemplary airway system 130, including a tracheal tube 132, shown here as endotracheal tube, with an inflatable balloon cuff 134 that may be inflated to form a seal against the tracheal walls 136. The system 130 may also include devices that facilitate positive pressure ventilation of a patient, such as a ventilator 138, which may include any ventilator, such as those available from Nellcor Puritan Bennett, LLC.

As shown, the cuff 134 is operatively connected to a cuff inflation device 140 that in turn communicates with a monitor 12. The monitor 12, based in information from the visualization device 14 (not shown), includes image data from the trachea that may be displayed to facilitate and estimate of the tracheal size. In one embodiment, the tracheal size may be estimated based on a selection of the best-fitting representative tracheal tube 64. The monitor 12 may provide information to the cuff inflation device 140 related to the estimated tracheal size. In one embodiment, standard cuff inflation volumes and pressures are based on the tracheal size of a typical patient. If the trachea is estimated to be larger than the estimates for the typical patient, the cuff inflation device 140 may change its set limits on cuff inflation volume or pressure to account for a larger cuff inflation volume. If the trachea is estimated to be smaller than the estimates for the typical patient, the cuff inflation device 140 may likewise account for a smaller inflation volume.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Indeed, the disclosed embodiments may not only be applied to the selection of tracheal tubes of a particular size relative to a visualized airway, but these techniques may also be utilized for the measurement and/or analysis of the placement of other suitable medical devices relative to other anatomical structures. For example, the present techniques may be utilized for the placement of tracheal tubes relative to the carina or other anatomical features. In addition, the present techniques may be employed in determining appropriate selection and placement of any medical device, such as a stent, catheter, implant, feeding tube, cardiac device, drug delivery device, or pump. Rather, the various embodiments may cover all modifications, equivalents, and alternatives falling within the spirit and scope of the disclosure as defined by the following appended claims.

What is claimed is:

1. A method for visualizing an airway of a patient comprising:
receiving image data from a device inserted into an airway of a patient;
displaying an airway image based on the image data;
accessing a representative image of a tracheal tube, wherein the representative image comprises one or more of a plurality of representative images of tracheal tubes of different sizes; and
superimposing the representative image onto the displayed airway image.

2. The method of claim 1, comprising removing the representative image from the display; and superimposing a second representative image of a tracheal tube onto the airway image based on a user input, wherein the second representative image is representative of a tracheal tube of a different size than the first representative image.

3. The method of claim 1, wherein the representative image is scaled to the airway image such that the representative image and the airway image are of approximately the same scale.

4. The method of claim 3, wherein the airway image is scaled to the representative image based on a user input.

5. The method of claim 1, comprising determining an airway diameter based on the airway image.

6. The method of claim 1, comprising determining an airway diameter based on a user input.

7. The method of claim 6, wherein the user input comprises a selection of a best fit from the plurality of representative images of tracheal tubes of different diameters.

8. The method of claim 1, comprising receiving a user input related to a position of the representative image relative to the airway image; and changing a position of the representative image of the tracheal tube relative to the airway image based on the user input.

9. A method for visualizing an airway of a subject comprising:
receiving image data from a device inserted into an airway of a patient, wherein the image data is associated with a plurality of locations within the airway;
receiving a user input related to one or more locations from the plurality of locations;
accessing a representative image of a tracheal tube, wherein the representative image comprises one or more images from a plurality of representative images of tracheal tubes of different shapes or sizes; and
superimposing said one or more representative images onto an airway image associated with one or more selected locations, wherein the one or more representative images are scaled to the airway image.

10. The method of claim 9, comprising determining the size or shape of the airway at the one or more selected locations based on the airway image.

11. The method of claim 9, comprising determining the size or shape of the airway at the one or more selected locations based on a second user input.

12. The method of claim 11, wherein the second user input comprises a selection of a best fit from the one or more images from the plurality of representative images of tracheal tubes.

13. The method of claim 9, wherein the one or more selected locations are associated with an anatomical landmark.

14. The method of claim 13, wherein the anatomical landmark comprises a narrowing of the airway.

15. The method of claim 9, wherein the one or more selected locations are representative of a smallest diameter region of the airway among the plurality of locations for which image data is received.

16. A device for visualizing an airway of a patient comprising:
a processor configured to:
receive image data from a device inserted into an airway of a patient;
display an airway image based on the image data; and
access a representative image of a tracheal tube, wherein the representative image is selected from a plurality of representative images of tracheal tubes of different diameters;
superimpose the representative image onto the displayed airway image, wherein the representative image is scaled to the airway image; and
receive user input related to the airway image or the representative image.

17. The device of claim 16, wherein user input comprises changing a position of the representative image relative to the airway image.

18. The device of claim 16, wherein the processor is configured to provide a recommendation for a tracheal tube size based on the diameter of the airway.

19. The device of claim 18, wherein the processor is configured to determine a diameter of the airway based on a user input related to the relationship between the representative image and the airway image.

20. The device of claim 18, wherein the processor is configured to store the airway image with the superimposed representative image.
